SEQUENCE LISTING (3 sequences)

SEQ ID NO: 1 - protein, 29 aa, Pseudhymenochirus merlini
IKIPSFFRNILKKVGKEAVSLMAGALKQS

SEQ ID NO: 2 - protein, 29 aa, Pseudhymenochirus merlini
IKIPSFFRNILKKVGKEAVSLIAGALKQS

SEQ ID NO: 3 - protein, 27 aa, Pseudhymenochirus merlini
GIFPIFAKLLGKVIKVASSLISKGRTE

(19) United States
(12) Patent Application Publication
(10) Pub. No.: US 2017/0209534 A1
(43) Pub. Date: Jul. 27, 2017

(54) Pseudhymenochirins As Agents For Treatment Of Cancer And Microbial Infections

(71) Applicant: United Arab Emirates University, Al Ain (AE)

(72) Inventors: John Michael Conlon, Omaha, NE (US); Milena Mechkarska, Sofia (BG)

(73) Assignee: United Arab Emirates University, Al Ain (AE)

(21) Appl. No.: 15/500,443

(22) PCT Filed: Jul. 30, 2014
(86) PCT No.: PCT/IB2014/063539

§ 371 Date: Jan. 30, 2017

(51) Int. Cl. A61K 38/17; A61K 9/00

(52) U.S. Cl. CPC A61K 38/1703; A61K 9/0014

(57) ABSTRACT

The invention relates to the use of pseudhymenochirin peptides as agents for the treatment of cancer and for the treatment of infections caused by multidrug-resistant microorganisms in cancer patients.

### FIELD OF THE INVENTION

This invention relates to the use of pseudhymenochirin peptides as anti-cancer agents and as anti-microbial agents.

### BACKGROUND

Cancer is a group of diseases which is manifested by the abnormal cell growth or immortality of the cells in a body. These immortal cells do not possess contact inhibition and are capable of migrating to and invading different parts of the body through body fluids.

Currently, cancer is treated using any or all of the techniques like chemotherapy, radiotherapy and surgery. The development of resistance to commonly used anticancer agents poses serious problems in cancer chemotherapy. Naturally occurring host-defence peptides, and analogues that show selective cytotoxicity against tumour cells, have potential for development into anti-cancer agents in cases wherein, the tumour is not responsive to conventional therapies (Schweizer, 2009).

The emergence in all regions of the world of strains of pathogenic bacteria and fungi with resistance to commonly used antibiotics constitutes a serious threat to public health. Of particular concern is the appearance of strains of the Gram-positive bacterium Staphylococcus aureus that are resistant to all beta-lactam antibiotics. Generally referred to as methicillin-resistant S. aureus (MRSA), these strains often exhibit multi-drug resistance, including non-susceptibility to quinolones, macrolides and sulfonamides (Gould et al., 2012). Although effective new types of antibiotics against multidrug-resistant Gram-positive bacteria such as MRSA have been introduced or are in clinical trials, the situation regarding new treatment options for infections produced by multidrug-resistant Gram-negative pathogens is less encouraging (Giamarellou et al., 2009; Nordmann et al., 2011; Savard et al., 2012). Similarly, multidrug-resistant strains of the Gram-negative bacterium Acinetobacter baumannii (MDRAB) are responsible for a range of infections that are typically encountered in immune-compromised and critically ill patients in intensive-care units (Dijkshoorn et al., 2007). Also, cancer patients are prone to secondary infections. Watanabe et al, 1992, mention that S. aureus, including MRSA, Klebsiella spp. and Pseudomonas aeruginosa are the major causative pathogens in lung cancer patients. Thus, there is a constant need for new types of agents with appropriate pharmacokinetic and toxicological profiles that are active against these antibiotic-resistant microorganisms.

Peptidomic analysis of skin secretions from Merlin's clawed frog Pseudhymenochirus merlini led to purification and characterization of 13 host-defence peptides with antimicrobial activity (Conlon et al., 2013). Preliminary data indicated that endogenous pseudhymenochirin-1Pb (Ps-1Pb) and pseudhymenochirin-2 Pa (Ps-2 Pa) inhibited the growth of reference strains of the bacteria Escherichia coli and S. aureus (Conlon et al., 2013) but otherwise their biological properties have not been substantively investigated.

The invention addresses the problems mentioned in the prior art and provides an anti-cancer agent and an anti-microbial agent.

### SUMMARY OF THE INVENTION

The present invention relates to a method of treating cancer and/or inhibiting the growth of cancer cells using peptides belonging to the pseudhymenochirin family.

Accordingly, one aspect of the invention provides for a method of treating cancer using pseudhymenochirin-1Pb or pseudhymenochirin-2 Pa. In an embodiment of the invention, the pseudhymenochirins are used for the treatment of lung cancer, breast cancer or colorectal cancer.

In another embodiment, a pseudhymenochirin peptide may be used in combination with additional therapeutic agent for the treatment of cancer.

The peptides belonging to the pseudhymenochirin family are capable of stimulating immunomodulatory cytokines. In particular, the peptides stimulate production of pro-inflammatory cytokines, and suppress the production of anti-inflammatory cytokines.

Another aspect of the invention is a method of treating microbial infections comprising administering an effective amount of a pseudhymenochirin peptide.

In an embodiment, the invention claims a method of treating multidrug-resistant microbial infection using pseudhymenochirin-1Pb or pseudhymenochirin-2 Pa.

In yet another embodiment, the invention claims a method of treating secondary microbial infections in a subject with cancer using pseudhymenochirin-1Pb or pseudhymenochirin-2 Pa.

In a preferred embodiment, the pseudhymenochirin peptide is topically applied or administered to treat microbial infection. In particular, the microbial infection may be a microbial skin infection.

A yet another aspect of the invention is a pseudhymenochirin peptide for use in the treatment of cancer. In a preferred embodiment, pseudhymenochirin-1Pb or pseudhymenochirin-2a are used in the treatment of cancer.

### BRIEF DESCRIPTION OF THE FIGURES

FIG. 1 Effects of pseudhymenochirin-1Pb on the viability of (A) non-small cell lung adenocarcinoma A549 cells, (B) breast adenocarcinoma MDA-MB-231 cells, (C) colorectal adenocarcinoma HT-29 cells, and (D) umbilical vein HUVEC cells after 24 h exposure. Columns: mean; bars: SEM.

FIG. 2 Effects of pseudhymenochirin-2 Pa on the viability of (A) non-small cell lung adenocarcinoma A549 cells, (B) breast adenocarcinoma MDA-MB-231 cells, (C) colorectal adenocarcinoma HT-29 cells, and (D) umbilical vein HUVEC cells after 24 h exposure. Columns: mean; bars: SEM.

FIG. 3 Effects of pseudhymenochirin-1Pb and pseudhymenochirin-2 Pa on the production of IL-10 by unstimulated and lipopolysaccharide (LPS)-stimulated mouse peritoneal macrophages. (*) P<0.05 v/s medium only.

FIG. 4 Effects of pseudhymenochirin-1Pb and pseudhymenochirin-2 Pa on the production of IL-23 by unstimulated and lipopolysaccharide (LPS)-stimulated mouse peritoneal macrophages. (*) P<0.05 v/s medium only.

FIG. 5 Effects of pseudhymenochirin-1Pb and psedhymenochirin-2 Pa on the production of IL-6 by unstimulated and lipopolysaccharide (LPS)-stimulated mouse peritoneal macrophages. (*) P<0.05 v/s medium only.

FIG. 6 Survival of (A) S. aureus (ATCC 25923) and (B) E. coli (ATCC 25726) in Mueller-Hinton broth after addition of 4×MIC psedhymenochirin-1Pb. Control represents incubation in the absence of peptide. CFU: colony forming units.

### DETAILED DESCRIPTION OF THE INVENTION

The present invention provides a method for treating cancer and a method for treating microbial skin infections in cancer subjects comprising administrating to a subject, a therapeutically effective amount of a pseudhymenochirin peptide.

Pseudhymenochirins are a group of cationic, α-helical, host-defence peptides, isolated from the skin secretion of the frog belonging to the genus Pseudhymenochirus and Pipidae family. The pseudhymenochirin peptides pseudhymenochirin-1 Pa, pseudhymenochirin-1Pb and pseudhymenochirin-2 Pa are known. Pseudhymenochirin-1 Pa and pseudhymenochirin-1Pb differ in their peptide sequence by a single amino acid. Pseudhymenochirin-1 Pa has the peptide sequence IKIPSFFRNILKKVGKEAVSLMAGALKQS. Pseudhymenochirin-1Pb has the peptide sequence IKIPSFFRNILKKVGKEAVSLIAGALKQS. Pseudhymenochirin-2 Pa has the peptide sequence GIFPIFAKLLGKVIKVASSLISKGRTE. The preferred pseudhymenochirins used in accordance with this invention are pseudhymenochirin-1Pb and pseudhymenochirin-2 Pa.

The peptides of the present invention may be prepared using well known techniques. For example, the peptides of the present invention may be prepared by recombinant DNA technology or chemical synthesis. The peptides of the present invention may contain modifications, such as glycosylation, side chain oxidation, or phosphorylation, provided such modifications do not destroy the biological activity of the original peptides. Other illustrative modifications include incorporation of D-amino acids or other amino acid mimetics.

Without being bound by theory, pseudhymenochirins are able to treat cancer by stimulating the immunomodulatory cytokines. Pseudhymenochirins, such as pseudhymenochirin-1Pb and pseudhymenochirin-2 Pa, are capable of increasing the production of pro-inflammatory cytokines like interleukin-23 and decreasing the production of anti-inflammatory cytokine like interleukin-10. Pseudhymenochirins, such as pseudhymenochirin-1Pb and pseudhymenochirin-2 Pa, are also found to decrease the concentration of interleukin-6, which serves as both a pro-inflammatory and an anti-inflammatory cytokine. The peptides, by increasing the production of pro-inflammatory cytokines, may enhance the effect of innate immune response to tumorigenesis.

Accordingly, a further aspect of the invention comprises a method for altering immunomodulatory cytokine production comprising administering a pseudhymenochirin peptide to a subject in need thereof. In particular, the method is for increasing the production of pro-inflammatory cytokines and decreasing the production of anti-inflammatory cytokines.

The phrase “effective amount” indicates the amount of pseudhymenochirin peptide which is effective in treating any aspect of cancer or secondary microbial infections in cancer patients. A therapeutically effective amount can be given in one or more administrations.

The term “treating” is used conventionally, e.g., the management or care of the subject for the purpose of combating, alleviating, reducing, relieving, improving, etc., one or more of the symptoms associated with cancer or secondary microbial infections. Administering effective amounts of a pseudhymenochirin peptide may treat one or more aspects of the cancer disease, including, but not limited to, causing tumour regression; inhibiting cell proliferation; inhibiting tumour growth; causing cell death; causing necrosis; causing apoptosis; inhibiting tumour metastasis; inhibiting tumour migration; inhibiting tumour invasion; reducing disease progression; stabilizing the disease; reducing or inhibiting angiogenesis; and prolonging patient survival.

Examples of the cancer that may be treated with pseudhymenochirins peptides include, but are not limited to, Accelerated Phase Chronic Myelogenous Leukemia; Acute Erythroid Leukemia; Acute Lymphoblastic Leukemia; Acute Lymphoblastic Leukemia in Remission; Acute Lymphocytic Leukemia; Acute Monoblastic and Acute; Monocytic Leukemia; Acute Myelogenous Leukemia; Acute Myeloid Leukemia; Adenocarcinoma of the Prostate; Adenoid Cystic Carcinoma of the Head and Neck; Advanced Gastrointestinal Stromal Tumour; Agnogenic Myeloid; Metaplasia; Anaplastic Oligodendroglioma; Astrocytoma; B-Cell Adult Acute Lymphoblastic Leukemia; Blastic Phase Chronic Myelogenous Leukemia; Bone Metastases; Brain Tumour; Breast Cancer; Central Nervous System Cancer; Childhood Acute Lymphoblastic Leukemia; Childhood Acute Lymphoblastic Leukemia in Remission; Childhood Central Nervous System Germ Cell Tumour; Childhood Chronic Myelogenous Leukemia; Childhood Soft Tissue Sarcoma; Chordoma; Chronic Eosinophilic Leukemia (CEL); Chronic Idiopathic Myelofibrosis; Chronic Myelogenous Leukemia; Chronic Myeloid Leukemia; Chronic Myelomonocytic Leukemia; Chronic Phase Chronic Myelogenous Leukemia; Colon Cancer; Colorectal Cancer; Dermatofibrosarcoma; Dermatofibrosarcoma Protuberans (DFSP); Desmoid Tumour; Eosinophilia; Epidemic Kaposi's Sarcoma; Essential Thrombocythemia; Ewing's Family of Tumours; Extensive Stage Small Cell Lung Cancer; Fallopian Tube Cancer; Familiar Hypereosinophilia; Fibrosarcoma; Gastric Adenocarcinoma; Gastrointestinal Neoplasm; Gastrointestinal Stromal Tumour; Glioblastoma; Glioma; Gliosarcoma; Grade I Meningioma; Grade II Meningioma; Grade III Meningioma; Hematopoietic and Lymphoid Cancer; High-Grade Childhood Cerebral Astrocytoma; Hypereosinophilic Syndrome; Idiopathic Pulmonary Fibrosis; L1 Adult Acute Lymphoblastic Leukemia; L2 Adult Acute Lymphoblastic Leukemia; Leukemia, Lymphocytic, Acute L2; Leukemia, Myeloid, Chronic; Leukemia, Myeloid, Chronic Phase; Liver Dysfunction and Neoplasm; Lung Disease; Lymphoid Blastic Phase of Chronic Myeloid Leukemia; Male Breast Cancer; Malignant Fibrous Histiocytoma; Mastocytosis; Meningeal Hemangiopericytoma; Meningioma; Myelofibrosis; Myeloid Leukemia, Chronic; Myeloid Leukemia, Chronic Accelerated-Phase; Myeloid Leukemia, Chronic, Chronic-Phase; Myeloid Metaplasia; Myeloproliferative Disorder (MPD) with Eosinophilia; Neuroblastoma; Non-T, Non-B Childhood Acute Lymphoblastic Leukemia; Oligodendroglioma; Osteosarcoma; Ovarian Germ Cell Tumour; Ovarian Low Malignant Potential Tumour; Ovarian Neoplasms; Pancreatic Cancer; Pelvic Neoplasms; Peritoneal Cavity Cancer; Peritoneal Neoplasms; Philadelphia Chromosome Positive Chronic Myelogenous Leukemia; Philadelphia Positive Acute Lymphoblastic Leukemia; Philadelphia Positive Chronic Myeloid Leukemia in Myeloid Blast Crisis; Polycythemia Vera; Pulmonary Fibrosis; Recurrent Adult Brain Tumour; Recurrent Adult Soft Tissue Sarcoma; Recurrent Breast Cancer; Recurrent Colon Cancer; Recurrent Esophageal Cancer; Recurrent Gastric Cancer; Recurrent Glioblastoma Multiforme (GBM); Recurrent Kaposi's Sarcoma; Recurrent Melanoma; Recurrent Merkel Cell Carcinoma; Recurrent Ovarian Epithelial Cancer; Recurrent Pancreatic Cancer; Recurrent Prostate Cancer; Recurrent Rectal Cancer; Recurrent Salivary Gland Cancer; Recurrent Small Cell Lung Cancer; Recurrent Tumours of the Ewing's Family; Recurrent Uterine Sarcoma; Relapsing Chronic Myelogenous Leukemia; Rheumatoid Arthritis; Salivary Gland Adenoid Cystic Carcinoma; Sarcoma; Small Cell Lung Cancer; Stage II Melanoma; Stage II Merkel Cell Carcinoma; Stage III Adult Soft Tissue Sarcoma; Stage III Esophageal Cancer; Stage III Merkel Cell Carcinoma; Stage III Ovarian Epithelial Cancer; Stage III Pancreatic Cancer; Stage III Salivary Gland Cancer; Stage IIIB Breast Cancer; Stage IIIC Breast Cancer; Stage IV Adult Soft Tissue Sarcoma; Stage IV Breast Cancer; Stage IV Colon Cancer; Stage IV Esophageal Cancer; Stage IV Gastric Cancer; Stage IV Melanoma; Stage IV Ovarian Epithelial Cancer; Stage IV Prostate Cancer; Stage IV Rectal Cancer; Stage IV Salivary Gland Cancer; Stage IVA Pancreatic Cancer; Stage IVB Pancreatic Cancer; Systemic Mastocytosis; T-Cell Childhood Acute Lymphoblastic Leukemia; Testicular Cancer; Thyroid Cancer; Unresectable or Metastatic Malignant Gastrointestinal Stromal Tumour (GIST); Unspecified Adult Solid Tumour; Untreated Childhood Brain Stem Glioma; Uterine Carcinosarcoma, and Uterine Sarcoma. In particular, the peptides are useful in the treatment of lung, colorectal and breast cancers.

The term “subject” in accordance with the invention may relate to any mammals including, but not limited to humans, monkeys, non-human primates, mice, rats, dogs, and cats. In particular, the subject is a human.

Patients suffering from cancer are commonly co-administered with additional therapeutic agents, in particular further suitable antineoplastic or anti-tumour agents used include but are not limited to:

1. Alkylating antineoplastic agents: such as cisplatin, carboplatin, oxaliplatin, mechlorethamine, cyclophosphamide, chlorambucil, and ifosfamide.
2. Plant alkaloids and terpenoids. These include:
i. vinca alkaloids such as vincristine, vinblastine, vinorelbine and vindesine;
ii. podophyllotoxin, etoposide and teniposide; and
iii. taxanes, such as paclitaxel, originally known as taxol, and docetaxel.
   3. Topoisomerase inhibitors:
i. type I topoisomerase inhibitors include camptothecins: irinotecan and topotecan; and
ii. type II inhibitors include amsacrine, etoposide, etoposide phosphate and teniposide.
   4. Cytotoxic antibiotics such as actinomycin, anthracyclins, doxorubicin, daunorubicin, valrubicin and epirubicin. Other cytotoxic antibiotics include bleomycin, plicamycin and mitomycin.

Other therapeutic agents are also commonly administered to patients to deal with the side effects of chemotherapy. Such agents might include anti-emetics for nausea, or agents to treat anaemia and fatigue. Other such medicaments are well known to physicians and skilled in cancer therapy.

Such agents may be administered sequentially, simultaneously or concomitantly.

A further aspect of the invention provides a pharmaceutical composition comprising a pseudhymenochirin peptide and a pharmaceutically acceptable carrier and/or excipient for use in treating cancer and/or microbial infections.

Pharmaceutical forms suitable for the delivery of the compounds of the present invention and methods of preparing the various pharmaceutical compositions will be readily apparent to those skilled in the art.

The invention provides for a method of treating microbial infections using the pseudhymenochirin peptides, in particular pseudhymenochirin-1Pb and pseudhymenochirin-2a. In a preferred embodiment, the peptides are used to treat secondary microbial infections caused by multidrug-resistant microorganisms in subjects with cancer and/or undergoing cancer treatment.

The phrase “secondary microbial infections” refers to the microbial infections caused in cancer subjects as a result of low or diminished immune response due to conventional cancer treatment methodologies (chemotherapy or radiotherapy). These secondary microbial infections may include infections caused by and not limited to micro-organisms: Staphylococcus aureus, Klebsiella pneumoniae, Pseudomonas aeruginosa, Enterococcus faecalis, Candida albicans, Acinetobacter baumannii, Stenotrophomonas maltophilia and multidrug-resistant clinical isolates.

The phrase “multidrug-resistant microbial infections” refer to infections caused by the micro-organisms which are resistant to antibiotics which are generally used for their treatment e.g. beta-lactam antibiotics.

A further aspect of the invention provides a method of treating skin infections.

Skin infections, often in otherwise healthy young individuals, are a common clinical manifestation of community-acquired MRSA infections (Skov et al., 2012) and MRSA is a serious problem in infections of the superficial epidermis such as impetigo in infants and children (Geria et al., 2010) and in colonization of surface lesions such as the foot ulcers of diabetic patients (Bowling et al., 2009).

The high haemolytic activity of Ps-1Pb may limit its use as a systemic anti-infective agent. However, peptides administered to infected skin or skin lesions can penetrate into the stratum corneum to kill microorganisms. Therefore, in a preferred embodiment, the method of treating skin infections comprises topically administering a pseudhymenochirin peptide to a subject thereof. The Ps-1Pb and Ps-2 Pa may be administered in the form of a topical treatment for combating MRSA skin infections, decolonization of MRSA carriers, and in therapeutic regimes to promote wound healing.

Further aspects and embodiments of the invention include:

The use of a pseudhymenochirin peptide in the manufacture of a medicament for use in medicine.

The use of a pseudhymenochirin peptide in combination with a further therapeutic agent for use in medicine.

A pseudhymenochirin peptide for use in the treatment of cancer.

The use of a pseudhymenochirin peptide in the manufacture of a medicament for the treatment of cancer.

The use of a pseudhymenochirin peptide in combination with another therapeutic agent in the manufacture of a medicament for the treatment of cancer.

The use of a pseudhymenochirin peptide in combination with another therapeutic agent for the treatment of cancer.

The use of a pseudhymenochirin peptide for treating lung cancer, breast cancer and colorectal cancer.

A pseudhymenochirin peptide for use in the treatment of microbial infections.

A pseudhymenochirin peptide in combination with another therapeutic agent for use in the treatment of microbial infections.

The use of a pseudhymenochirin peptide in the manufacture of a medicament for the treatment of microbial infections.

The use of a pseudhymenochirin peptide in combination with another therapeutic agent in the manufacture of a medicament for the treatment of microbial infections.

A pseudhymenochirin peptide for use in treating microbial skin infection, multidrug-resistant microbial infection, secondary microbial infection, nosocomial infections, community-acquired infections and opportunistic microbial infections.

Preferably, the pseudhymenochirins for the above mentioned uses are selected from pseudhymenochirn-2 Pa and pseudhymenochirn-1Pb.

It must be noted that as used herein, the singular forms “a”, “and”, and “the” include plural referents unless the content clearly dictates otherwise.

The invention will now be described with reference to the following examples which are provided to illustrate, but not to limit the invention.

### Examples

### Abbreviations

Ps-1Pb, Pseudhymenochirin-1Pb; Ps-2 Pa, Pseudhymenochirin-2 Pa; RP-HPLC, reversed phase High-performance liquid chromatography; FCS, Fetal Calf Serum; HUVEC, Human Umbilical Vein Endothelial Cells; RPMI medium, Roswell Park Memorial Institute medium; DMEM, Dulbecco's Modified Eagle's Medium; ATP, Adenosine Tri-Phosphate; RBC, Red Blood Cells; DPBS, Dulbecco's phosphate-buffered saline; LPS, Lipo-polysaccharide; ELISA, Enzyme linked immune-sorbent assay; MRSA, Methicillin-resistant Staphylococcus aureus; CFU, Colony forming units; MIC, Minimum inhibitory concentration; and MDRAB, Multidrug-resistant Acinetobacter baumannii.

### Materials and Methods

### Peptides, Reagents and Cell Culture

Ps-1Pb (IKIPSFFRNILKKVGKEAVSLIAGALKQS) and Ps-2 Pa (GIFPIFAKLLGKV IKVASSLISKGRTE) were supplied in crude form by GL Biochem Ltd (Shanghai, China); FCS was supplied from Biowest, France; EndoGRO MV-VEGF Complete Media Kit was supplied from Millipore, USA; CellTiter-Glo Luminescent Cell Viability assay from Promega Corporation, USA; LPS from E. coli 055:65 was sourced from Sigma-Aldrich, USA; ELISA assay kits from R & D Systems, USA; and Microbial cultures S. aureus (ATCC 25923), E. coli (ATCC 25726), K. pneumoniae (ATCC 700603), P. aeruginosa (ATCC 27853), Enterococcus faecalis (ATCC 29212) and Candida albicans (ATCC 90028) were obtained from American Type Culture Collection (ATCC), USA.

### Culture Media

### Mueller-Hinton Broth

Beef infusion—30.0%

Casein hydrolysate—1.75%

Starch—0.15%

pH adjusted to neutral at 25° C.

### Peptide Synthesis

The peptides (Ps-1Pb and Ps-2 Pa) were supplied in crude form and were purified by RP-HPLC using a Vydac 218TP1022 (C-18) column with the column area 2.2 cm×25 cm. Prior to its use, the column was equilibrated with acetonitrile/water/trifluoroacetic acid mixture at the ratio 35.0/64.9/0.1 v/v/v at a constant flow rate of 6 mL/min. The concentration of acetonitrile was raised to 63% (v/v) over 60 min using a linear gradient. Absorbance was then measured at 214 nm and 280 nm and the major peak in the chromatogram was collected manually.

The final purity of the peptides was greater than 98% purity as determined by symmetrical peak shape and electro-spray mass spectrometry.

The obtained purified peptides showed high solubility in physiological buffers.

### Cytotoxicity Assays

For cytotoxicity assay, the effect of the peptides on three different types of cancer cells were analysed, human non-small cell lung adenocarcinoma A549 cells, human breast adenocarcinoma MDA-MB-231 cells and human colorectal adenocarcinoma HT-29 cells. As control, HUVEC cell line was selected.

A549 cell line was maintained in RPMI 1640 medium containing 2 mM L-glutamine and supplemented with 10% FCS at 37° C. MDA-MB-231 and HT-29 cell lines were maintained in DMEM medium supplemented with 10% FCS at 37° C. The medium for cancer cells were further supplemented with antibiotics, penicillin 50 U/mL and streptomycin 50 μ/mL. HUVEC cells were maintained in EndoGRO MV-VEGF Complete Media Kit at 37° C.

After the cells have been stably maintained in their growth mediums, the viability of the cell lines were tested at multiple intervals and after individual passages using trypan blue dye exclusion test. The cancer cell lines and HUVEC cell line in their respective mediums showed viability of more than 99%.

### Viability Assay

The cells from each of the four cell lines were seeded individually in a 96-wells plate at a cell density of 5×10³ cells/well. After 24 hrs, the cells were treated with increasing concentrations of Ps-1Pb and Ps-2 Pa in the range of 1 μM-100 μM in triplicates.

The effect of the peptides on the cell viability was determined by the measurement of the concentration of ATP using CellTitre-Glo Luminescent Cell Viability assay. Luminescent signals were measured using a GLOMAX Luminometer system.

The LC₅₀ value was calculated for both the peptides for individual cell lines by conducting non-linear regression analysis using commercially available software (SPPS version 17.0; SPS Inc, Chicago, Ill., USA). The LC₅₀ value was calculated as the mean concentration of peptide producing 50% cell death.

### Haemolysis Assay

For analysis of the haemolytic activity, RBCs (2×10⁷ cells) taken from a healthy donor in DBPS, pH 7.4 (100 μL) were incubated with Ps-1Pb and Ps-2 Pa respectively in varying concentrations ranging from 1.6 μM to 100 μM of the peptides for a period of 1 hour at 37° C. After 1 hr, the samples were centrifuged at 12000 rpm for 15 seconds and the supernatant was collected. Absorbance of the supernatant was measured at 450 nm.

As control, the RBCs at a concentration of 2×10⁷ were incubated in DBPS as a negative control to detect the absorbance in the absence of haemolysis. In addition, positive control was also maintained in which the RBC at a concentration of 2×10⁷ was incubated with DPBS and 1% v/v Tween-20. The controls were centrifuged at 12000 rpm for 15 seconds and an absorbance reading was taken for the supernatant at 450 nm.

The LC₅₀ value was taken as the mean concentration of peptide producing 50% haemolysis in three independent experiments.

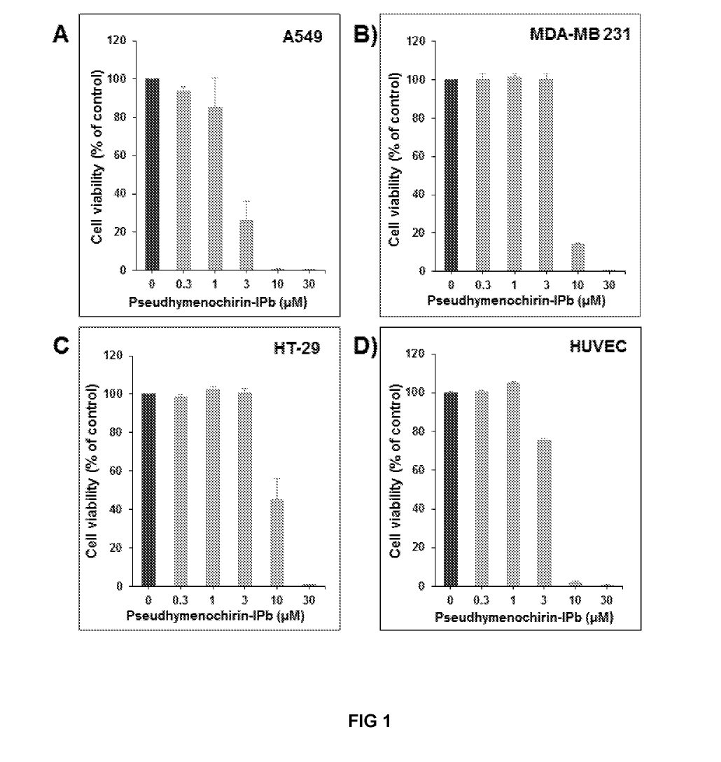

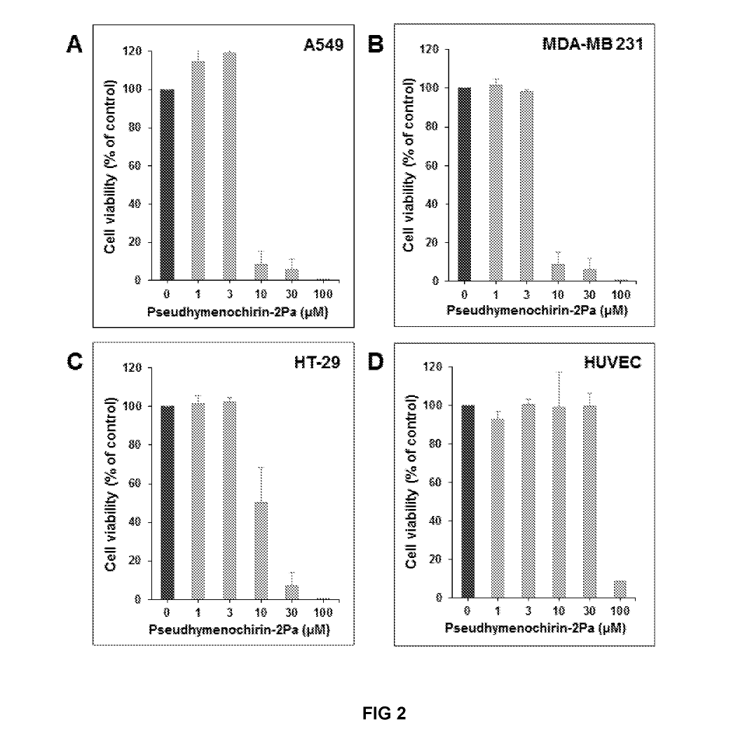

The results of the cytotoxicity and haemolysis assays are shown in Tables 1 and 2 and FIGS. 1 and 2.

The abilities of Ps-1Pb and Ps-2 Pa to produce cell death in three established human tumour cell lines during 24 hrs incubation are compared with their cytotoxic activities against non-neoplastic HUVEC cells and human red blood cells in Table 1.

TABLE 1
Cytotoxicity of pseudhymenochirin-1Pb and pseudhymenochirin-2Pa against
lung adenocarcinoma A549 cells, breast adenocarcinoma MDA-MB-231 cells,
colorectal adenocarcinoma HT-29 cells, HUVEC cells, and human red blood cells (RBC).

| | A549 | MDA-231 | HT-29 | HUVEC | RBC |
|---|---|---|---|---|---|
| Pseudhymenochirin-1Pb | 2.5 ± 0.2 | 6.6 ± 0.3 | 9.5 ± 1.3 | 5.6 ± 0.9 | 28 ± 2 |
| Pseudhymenochirin-2Pa | 6.0 ± 0.6 | 6.2 ± 0.6 | 11.5 ± 2.6 | 68 ± 2 | 6.2 ± 1.0 |

Data show mean LC₅₀ values (μM) ± S.E.M.

Both peptides (Ps-1Pb and Ps-2 Pa) exhibited cytotoxicity against human non-small cell lung adenocarcinoma A549 cells, breast adenocarcinoma MDA-MB-231 cells, and colorectal adenocarcinoma HT-29 cells with mean LC₅₀ values 12 μM.

As shown in FIGS. 1 and 2, A549 cells were the most sensitive to the cytotoxic action of the peptides and HT-29 cells the most resistant. Ps-1Pb showed no selectivity for tumour cells as the LC50 against non-neoplastic HUVEC cells was in the same range as the values against cancer cells. However, the peptide was between 3- and 11-fold less cytotoxic to human erythrocytes as compared to the tumour cells. In contrast, Ps-2 Pa was strongly haemolytic against erythrocytes (LC₅₀=6 μM) but was appreciably less cytotoxic against HUVEC cells (LC50=68 μM).

The observation that both the peptides Ps-1Pb and Ps-2 Pa show potent in vitro cytotoxic activity against a range of human tumour cell lines indicate that both the peptides can be used as anti-cancer agents.

The therapeutic indices for the peptides Ps-1Pb and Ps-2 Pa are calculated as the ratio of LC₅₀ for HUVEC cells to the LC₅₀ for tumour cells.

TABLE 2
Therapeutic indices for pseudhymenochirin-1Pb
and pseudhymenochirin-2Pa

| | A549 | MDA-231 | HT-29 |
|---|---|---|---|
| Pseudhymenochirin- | 2.2 | 0.8 | 0.6 |
| 1Pb | | | |
| Pseudhymenochirin- | 11.3 | 11.0 | 5.9 |
| 2Pa | | | |

As seen in the table 1 and FIGS. 1 and 2, Ps-1Pb is significantly more potent than Ps-2 Pa against lung adenocarcinoma A549 cells and is less haemolytic. However, as the effect of Ps-1Pb on the cancer cells is same as that on the non-neoplastic HUVEC cells, it can be inferred that Ps-1Pb shows no or minimum selectivity against cancer cells. On the other hand, Ps-2 Pa is about 6- and 11-fold less potent against non-neoplastic HUVEC cells as compared to cancer cells. Thus, it may be inferred that Ps-2 Pa is selective for tumour cells, but has the disadvantage of appreciable haemolytic activity against human erythrocytes (RBCs).

### Cytokine Measurement

Experiments on the effect of the peptides on the production of cytokines were conducted using macrophages obtained from the peritoneal cavity from C57BL/6 mice (Pantic et al., 2014). The peritoneal cavity of the mice was washed with 5 mL of phosphate buffered saline, pH 7.4. The cells obtained were placed on petri dishes and incubated in RPMI-1640 for two hours at 37° C. After two hours, the non-adherent cells were discarded by simply titling the petri-dish and washing slowly with phosphate buffered saline with pH 7.4.

The adherent cells were obtained by vigorous washing with ice-cold culture medium. In this case, RPMI 1640 containing 10% heat-inactivated FCS, 2 mM L-glutamine, 100 IU/mL penicillin G and 100 μg/mL streptomycin was used.

To analyse the effect of the peptides on peritoneal macrophages, peritoneal macrophages (2×10⁵ cells/well) were incubated with peptides at the peptide concentration of 5, 10 and 20 μg/mL, with or without the presence of LPS from E. coli 055:B5. Cells were then cultured for 24 hrs at 37° C. in a supplemented culture medium in an humidified atmosphere of 5% CO₂-95% air. After incubation, cell-free supernatants were collected and analysed for the presence of cytokines.

Concentrations of the cytokines, interleukin-1β (IL-1β), interleukin-6 (IL-6), interleukin-10 (IL-10), interleukin-23 (IL-23), and tumour necrosis factor-α (TNF-α) were determined in triplicate using ELISA assay kits.

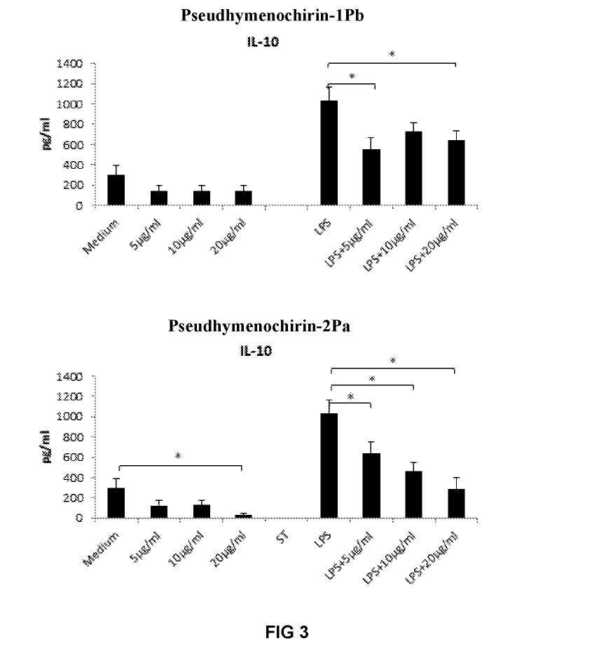

The results can be found in FIGS. 3-5.

The effects of Ps-1Pb and Ps-2 Pa, alone or together with a sub-maximal stimulatory dose of LPS (0.5 μg/mL), on cytokine production by peritoneal macrophages from C57BL/6 mice are shown in FIGS. 3-5. Ps-1Pb produced significant inhibition of IL-10 production only in LPS-stimulated cells. Ps-2 Pa produces a dose-dependent inhibition of the anti-inflammatory and immunosuppressive cytokine IL-10 by LPS-stimulated cells and significantly inhibited its production by unstimulated cells at a concentration of 20 μg/ml. The production of the pro-inflammatory cytokine IL-23 by both unstimulated and LPS-stimulated macrophages was significantly increased by incubation with both peptides at all tested doses (5, 10, and 20 μg/mL). Production of IL-6 by both unstimulated and LPS-stimulated cells was found to be strongly and dose-dependently inhibited by incubation with Ps-2 Pa. IL-6 production by unstimulated and LPS-stimulated cells was also significantly inhibited by Ps-1Pb at the highest dose tested (20 μg/ml). There was no significant effect on the production of the pro-inflammatory cytokines IL-1β and TNF-α by incubation with either of the peptides at any dose tested in both unstimulated and LPS-stimulated macrophages.

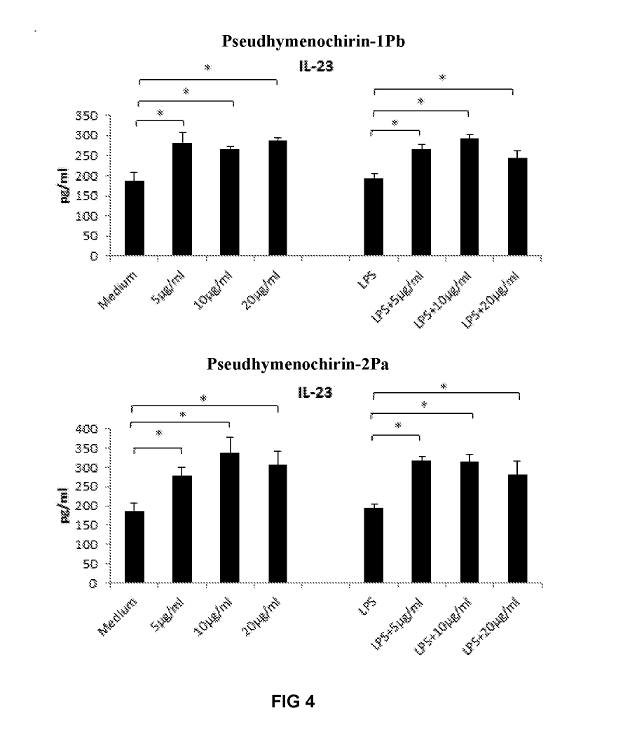

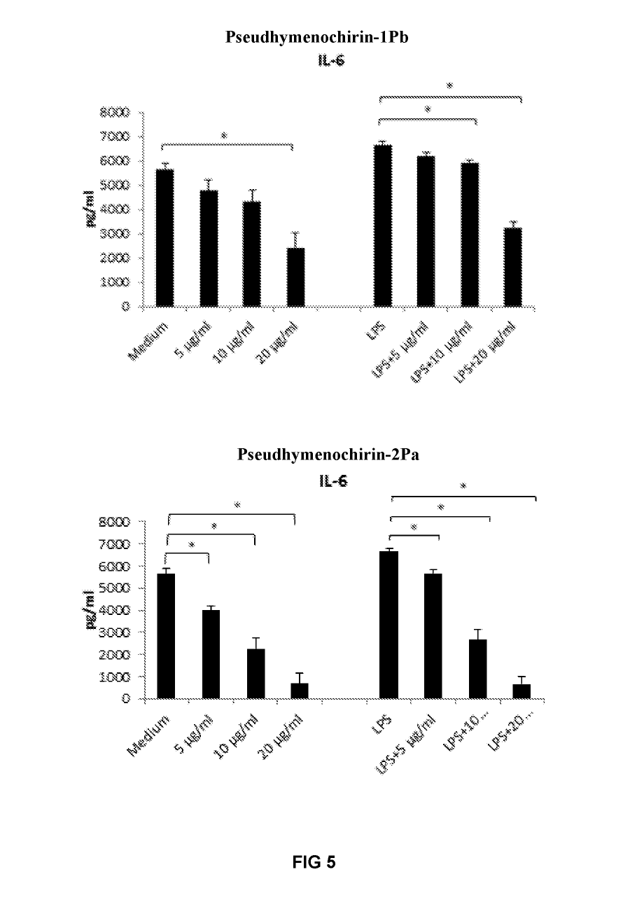

Ps-1Pb and Ps-2Pb potently stimulate IL-23 production (FIG. 5) while inhibiting production of IL-10 (FIG. 3) and IL-6 (FIG. 4). These peptides have no significant effect on the production of TNF-α and IL-1β.

The important immunomodulatory role played by peptides at the interface between innate and adaptive immunity is becoming appreciated. Pro-inflammatory cytokines function as immunostimulatory agents and several compounds that stimulate cytokine release are in clinical practice (Landry et al., 2008). Although generation of high levels of pro-inflammatory cytokines will lead to toxicity, enhancing their selective release provides a therapeutic application for Ps-1Pb and Ps-2 Pa. Without being bound by theory, the peptides, Ps-1Pb and Ps-2 Pa may have an enhancing effect on the innate immune response to tumorigenesis. This scenario could be beneficial in a clinical setting in which the first line of defence may be enhanced without possible complications due to induction of autoimmunity. Such peptides may provide a new class of immunostimulatory molecules to be used in combination with other anti-cancer drugs.

The ability of Ps-1Pb and Ps-2 Pa to increase production and release of IL-23 is complemented by suppression of the production of IL-10, a cytokine which down-regulates immune response. This effect may further enhance the immunostimulatory action of the peptide. Cancer cells synthesize and release factors such as IL-10 that promote immune tolerance to escape host immune surveillance. Elevated circulating concentrations of IL-10 have been measured in patients with a wide range of tumours and serum IL-10 levels correlate with tumour progression and the presence of metastases (Galizia et al., 2002). Consequently, agents that inhibit IL-10 release may enhance the efficacy of other forms of chemotherapy for the treatment of certain malignancies (Sredni 2012).

IL-6 is a multifunctional cytokine involved in the development of both Th1 and Th2 immune response based on different molecular mechanisms (Diehl et al., 2002). Although IL-6 is considered primarily as a pro-inflammatory cytokine, this cytokine can mediate immunosuppressive functions by a mechanism that involves the inhibition of NFκB binding activity followed by induction of the “classical” anti-inflammatory cytokine IL-10 (Hegde et al., 2004). Consequently, the ability of the pseudhymenochirins to inhibit the release of IL-6, as well as IL-10, from peritoneal cells, in concentrations as low as 5 μg/mL enhances their therapeutic potential as an anticancer agents.

### Microbial Assays

### Isolation of Organisms

Six clinical sporadic isolates of MRSA were isolated from the wounds of patients admitted to Tawam Hospital (Al Ain, UAE). The strains were characterised by multi-locus sequence typing (MLST), Staphylococcal cassette chromosome (SCCmec) typing, accessory gene regulator (agr) typing, Staphylococcus protein A (spa) typing, and toxin gene carriage (Sonnevend et al., 2012). The isolated strains (127/08, 145/08, 274/08, V4180, S908, and T4/6) were resistant to all β-lactam antibiotics tested and to a range of non-β-lactam antibiotics.

Acinetobacter strains (NM8, NM35, NM75, NM109, and NM124) were isolated from different hospitals in the UAE. The Acinetobacter strains were characterised based on PCR analysis. All the isolates were positive for bla_{OXA-51} gene. Their clonal lineage are Euro clone I (NM8 and NM75), Euro clone II (NM35 and NM109) or non-typeable (NM124). These bacteria were resistant to all antibiotics commonly used to treat Acinetobacter infections including cephalosporins, carbapenems, fluroquinolones, and aminoglycosides but were found sensitive to tigecycline and colistin (Mechkarska et al., 2013). Stenotrophomonas maltophilia strains (B32/1, B32/4, B5/5, B6/2, and U8708) were isolated from patients with bloodstream infection as discussed in Jumaa et al, 2006. The Stenotrophomonas isolates exhibited different molecular fingerprints by pulsed-field gel electrophoresis. All Stenotrophomonas strains were resistant to meropenem (MIC>32 mg/L) but were sensitive to co-trimoxazole (MIC<5 mg/L).

### Assay

Minimum Inhibitory Concentration (MIC) of the peptides was determined in duplicate in three independent experiments by standard micro-dilution methods according to CLSI guidelines (Clinical Laboratory and Standards Institute 2008a, 2008b) using 96-well microtitre cell-culture plates.

For bacteria assays, serial dilutions of the peptides (0.6-80 μM) in Mueller-Hinton broth (50 μL) were mixed with an inoculum of 50 μL of 10⁶ colony forming units (CFU)/mL) from a log-phase culture. The peptide and inoculum mixture was incubated for 18 h at 37° C. in a humidified atmosphere. For yeast, an inoculum of 5×10⁴ CFU/mL from Candida albicans was incubated in the presence of serial dilutions of the peptides (0.6-80 μM) in RPMI 1640 medium for 48 h at 35° C. After incubation, the absorbance at 630 nm of each well was determined using a microtiter plate reader.

As a control, incubations were carried out in parallel with increasing concentrations of antibiotics wherever the isolate was originally susceptible (ampicillin for S. aureus and E. coli, ciprofloxacin for K. pneumoniae and P. aeruginosa, and amphotericin B for C. albicans).

The results are shown in Tables 2 and 3.

TABLE 2
Minimum inhibitory concentrations (in μM) of pseudhymenochirin-1Pb
and pseudhymenochirin-2a against reference strains and
multidrug-resistant clinical isolates of Gram-positive bacteria
S. aureus

| | ATCC | | | | | | |
|---|---|---|---|---|---|---|---|
| | 25923 | 127/08 | 145/08 | 274/08 | S908 | T4/6 | V4180 |
| Pseudhymenochirin- | 5 | 5 | 10 | 5 | 5 | 5 | 5 |
| 1Pb | | | | | | | |
| Pseudhymenochirin- | 5 | 5 | 10 | 10 | 10 | 10 | 10 |
| 2Pa | | | | | | | |

S. epidermidis

| | RP62A | RP62A/1 | T6/19 | T37/8 |
|---|---|---|---|---|
| Pseudhymenochirin- | 2.5 | 1.25 | 2.5 | 2.5 |
| 1Pb | | | | |
| Pseudhymenochirin- | 5 | 2.5 | 5 | 10 |
| 2Pa | | | | |

E. faecalis

| | ATCC 29212 |
|---|---|
| Pseudhymenochirin-1Pb | 10 |
| Pseudhymenochirin-2Pa | 20 |

As seen in Table 2, the potencies of the two peptides were in the range of 1.25-10 μM for all the tested gram positive bacterial reference and MRSA strains.

The antimicrobial potencies of the peptides against reference stains of the clinically relevant Gram-negative bacteria E. coli, K. pneumoniae, and P. aeruginosa, and multidrug-resistant strains of A. baumannii, and S. maltophilia are shown in Table 3.

TABLE 3
Minimum inhibitory concentrations (in μM) of pseudhymenochirin-1Pb
and pseudhymenochirin-2Pa against reference strains and
multidrug-resistant clinical isolates of Gram-negative bacteria
and the opportunistic yeast pathogen C. albicans.
Reference strains

| | | K. | P. | |
|---|---|---|---|---|
| | E. coli | pneumoniae | aeruginosa | C. albicans |
| Pseudhymenochirin- | 10 | 20 | 20 | 80 |
| 1Pb | | | | |
| Pseudhymenochirin- | >80 | >80 | >80 | 80 |
| 2Pa | | | | |

Clinical isolates
A. baumannii

| | NM8 | NM35 | NM75 | NM109 | NM124 |
|---|---|---|---|---|---|
| Pseudhymenochirin- | 10 | 5 | 5 | 5 | 5 |
| 1Pb | | | | | |
| Pseudhymenochirin- | 20 | 10 | 10 | 10 | 20 |
| 2Pa | | | | | |

S. maltophilia

| | B32/1 | B32/4 | U8908 | B5/5 | B6/2 |
|---|---|---|---|---|---|
| Pseudhymenochirin- | 5 | 5 | 5 | 10 | 5 |
| 1Pb | | | | | |
| Pseudhymenochirin- | 10 | 10 | 20 | 40 | 20 |
| 2Pa | | | | | |

Ps-1Pb showed potent growth inhibitory activity against the clinical isolates of A. baumannii, and S. maltophilia (MIC in the range 5-10 μM). Ps-2 Pa showed consistently lower potency than Ps-1Pb against these clinical isolates (between 2- and 4-fold) and the peptide was not active against E. coli, K. pneumoniae, and P. aeruginosa at concentrations up to 80 μM.

Accordingly, it may be inferred that Ps-1Pb and Ps-2 Pa exhibited a potent growth inhibitory activity against a range of well-characterized clinical isolates of MRSA and MDRAB.

### Time Kill Assay

In order to determine the rate at which the peptides caused cell death, log-phase cultures of S. aureus (ATCC 25923) and E. coli (ATCC 25726) at a concentration of 0.5×10⁶ CFU/mL in Mueller-Hinton broth (1 ml) were incubated in polypropylene tubes at 37° C. with Ps-1Pb at a concentration of 4×MIC. Control incubations in the absence of peptide were also carried out. Aliquots of 25 μl were removed at the time intervals 0, 0.5, 1, 2, 3, 4 and 24 hrs and the CFU of the serially diluted samples was determined on Mueller-Hinton agar plates. The time required for 99.9% cell death was determined.

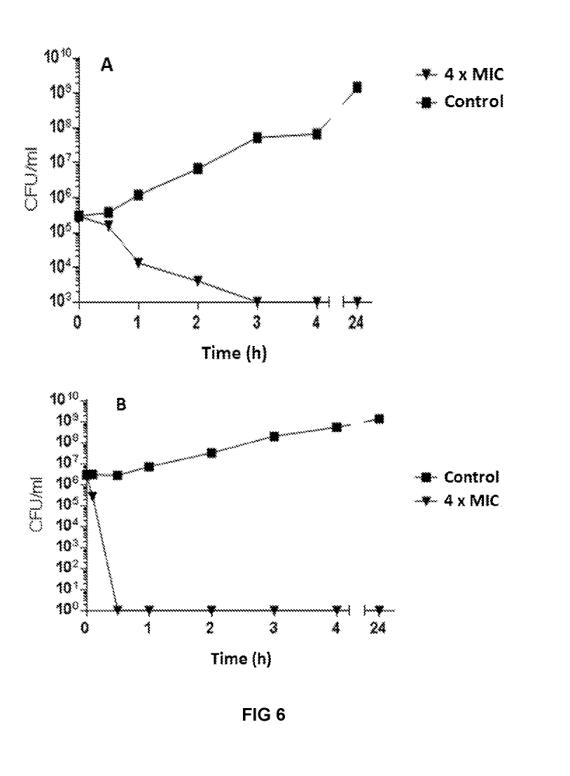

The results are shown in FIG. 6.

In a time-kill assay, incubation of Ps-1Pb at a concentration of 4×MIC with a reference strain of S. aureus resulted in killing of >99.9% of the bacteria within 180 min and on incubation of Ps-1Pb with a reference strain of E. coli under the same conditions resulted in 99.9% cell death within 30 min.

### Analysis of Results

Statistical analyses were performed using commercially available software (SPSS version 13.0; SPSS inc., Chicago, Ill., USA). The distributions of data were evaluated for normality using Kolmogorov-Smirnov test and then retested with Chi-Square test. Comparison of quantitative parametric data between two study groups was done by application of unpaired t-test. Differences between the paired data were evaluated using the paired t-test. With nonparametric data and two study groups Mann Whitney test was used. A p-value <0.05, from the two-sided tests, was considered statistically significant.

While the present invention has been described with respect to specific examples, it should be appreciated that the present invention is not limited to these examples. It is to be believed that one skilled in art, using the preceding description, can utilize the present invention to its fullest extent, and many variations and modifications may present themselves to those of skill in the art without diverting from the scope of the present invention.

All publications cited herein are hereby incorporated by reference in its entirety.

### CITATIONS

1. Bowling F L, Jude E B, Boulton A J. 2009. MRSA and diabetic foot wounds: contaminating or infecting organisms? Curr Diab Rep 9, 440-444;
2. Clinical Laboratory and Standards Institute. 2008a. Methods for dilution antimicrobial susceptibility tests for bacteria that grow aerobically. Approved Standard M07-A8. CLSI, Wayne Pa.;
3. Clinical Laboratory and Standards Institute. 2008b. Reference method for broth dilution antifungal susceptibility testing of yeast. Approved Standard M27-A3. CLS1, Wayne, Pa.;
4. Conlon J M, Prajeep M, Mechkarska M, Coquet L, Leprince J, Jouenne T, Vaudry H, King J D. 2013. Characterization of the host-defense peptides from skin secretions of Merlin's clawed frog Pseudhymenochirus merlini: insights into phylogenetic relationships among the Pipidae. Comp Biochem Physiol Part D Genomics Proteomics 8, 352-357;
5. Conlon J M, Mechkarska M. 2014. Host-defense peptides with therapeutic potential from skin secretions of frogs from the family Pipidae. Pharmaceuticals (Basel). 7, 58-77;
6. Conlon J M, Mechkarska M, Lukic M L, Flatt P R. 2014. Potential therapeutic applications of multifunctional host-defense peptides from frog skin as anti-cancer, anti-viral, immunomodulatory, and anti-diabetic agents. Peptides 57, 67-77;
7. Diehl S, Rincón M. 2002. The two faces of IL-6 on Th1/Th2 differentiation. Mol. Immunol 39, 531-536;
8. Dijkshoorn L, Nemec A, Seifert H. An increasing threat in hospitals: multidrug-resistant Acinetobacter baumannii. Nat Rev Microbiol 2007; 5:939-51;
9. Galizia G, Orditura M, Romano C, Lieto E, Castellano P, Pelosio L, Imperatore V, Catalano G, Pignatelli C, De Vita F. 2002. Prognostic significance of circulating IL-10 and IL-6 serum levels in colon cancer patients undergoing surgery. Clin Immunol 102, 169-178;
10. Geria A N, Schwartz R A. 2010. Impetigo update: new challenges in the era of methicillin resistance. Cutis 85, 65-70;
11. Giamarellou H, Poulakou G. 2009. Multidrug-resistant Gram-negative infections: what are the treatment options? Drugs 69, 1879-1901;
12. Gould I M, David M Z, Esposito S, Garau J, Lina G, Mazzei T, Peters G. 2012. New insights into meticillin-resistant Staphylococcus aureus (MRSA) pathogenesis, treatment and resistance. Int J Antimicrob Agents 39, 96-104;
13. Hegde S, Pahne J, Smola-Hess S. 2004. Novel immunosuppressive properties of interleukin-6 in dendritic cells: inhibition of NF-κB binding activity and CCR7 expression. FASEB J 18, 1439-1441;
14. Jumaa P A, Sonnevend A, Pál T, El Hag M, Amith R, Trad O. 2006. The molecular epidemiology of Stenotrophomonas maltophilia bacteraemia in a tertiary referral hospital in the United Arab Emirates 2000-2004. Ann Clin Microbiol Antimicrob 5, 32;
15. Landry Y, Gies J P. 2008. Drugs and their molecular targets: an updated overview. Fundam Clin Pharmacol 22, 1-18;
16. Mechkarska M, Prajeep M, Radosavljevic G D, Jovanovic I P, Al Baloushi A, Sonnevend A, Lukic M L, Conlon J M. 2013. An analog of the host-defense peptide hymenochirin-1B with potent broad-spectrum activity against multidrug-resistant bacteria and immunomodulatory properties. Peptides 50, 153-159;
17. Nordmann P, Naas T, Poirel L. 2011. Global spread of carbapenemase-producing Enterobacteriaceae. Emerg Infect Dis 17, 1791-1798;
18. Pantic J M, Mechkarska M, Lukic M L, Conlon J M. 2014. Effects of tigerinin peptides on cytokine production by mouse peritoneal macrophages and spleen cells and by human peripheral blood mononuclear cells. Biochimie 101, 83-92;
19. Savard P, Perl T M. 2012. A call for action: managing the emergence of multidrug-resistant Enterobacteriaceae in the acute care settings. Curr Opin Infec. Dis 25, 371-377.
20. Schweizer F. 2009. Cationic amphiphilic peptides with cancer-selective toxicity. Eur J Pharmacol 625, 190-194;
21. Skov R, Christiansen K, Dancer S J, Daum R S, Dryden M, Huang Y C, Lowy F D. Update on the prevention and control of community-acquired meticillin-resistant Staphylococcus aureus (CA-MRSA). Int J Antimicrob Agents 2012; 39:193-200;
22. Sonnevend À, Blair I, Alkaabi M, Jumaa P, Al Haj M, Ghazawi A, Akawi N, Jouhar F S, Hamadeh M B, Pal T. 2012. Change in meticillin-resistant Staphylococcus aureus clones at a tertiary care hospital in the United Arab Emirates over a 5-year period. J Clin Pathol 65, 178-82;
23. Sredni B. 2012. Immunomodulating tellurium compounds as anti-cancer agents. Semin Cancer Biol 22, 60-69; and
24. Watanabe A., Nakai Y., Saito J., Motomiya M., 1992, Respiratory infections associated with lung cancer, Gan To Kagaku Ryoho. February; 19(2):184-8.

1. A method of treating a cancer in a subject in need thereof, the method comprising administering an effective amount of a pseudhymenochirin peptide to the subject.

2. The method of claim 1, wherein the pseudhymenochirin peptide is selected from any of pseudhymenochirin-1 Pb and pseudhymenochirin-2 Pa.

3. The method of claim 1, wherein the pseudhymenochirin peptide is pseudhymenochirin-1 Pb.

4. The method of claim 1, wherein the pseudhymenochirin peptide is pseudhymenochirin-2 Pa.

5. The method of claim 1, wherein the cancer is selected from the group consisting of lung cancer, breast cancer and colorectal cancer.

6. The method of claim 1 further comprising administering the pseudhymenochirin peptide in combination with at least one additional therapeutic agent.

7. A method of treating at least one microbial infection in a subject in need thereof, the method comprising administering an effective amount of a pseudhymenochirin peptide to the subject.

8. The method of claim 7, wherein the subject has cancer.

9. The method of claim 7, wherein the microbial infection is caused by micro-organisms belonging to a genus selected from the group consisting of Staphylococcus, Enterococcus, Escherichia, Klebsiella, Pseudomonas, Candida, Acinetobacter, and Stenotrophomonas.

10. The method of claim 7, wherein the microbial infection is a multidrug-resistant microbial infection.

11. The method of claim 7, wherein the microbial infection is a microbial skin infection.

12. The method of claim 11, wherein the microbial skin infection is a methicillin-resistant Staphylococcus aureus (MRSA) skin infection.

13. The method of claim 7, wherein the pseudhymenochirin peptide is administered topically.

14. The method of claim 7, wherein the pseudhymenochirin peptide is selected from any of pseudhymenochirin-1 Pb and pseudhymenochirin-2 Pa.

15. The method of claim 7, wherein the pseudhymenochirin peptide is Pseudhymenochirin-1 Pb.

16. The method of claim 7, wherein the pseudhymenochirin peptide is Pseudhymenochirin-2 Pa.

17. A method of treating at least one secondary microbial infection in a subject in need thereof, the method comprising administering an effective amount of a pseudhymenochirin peptide to the subject.

18. The method of claim 17, wherein the subject has cancer.

19. A method of stimulating immunomodulatory cytokines in a subject in need thereof, the method comprising administering an effective amount of a pseudhymenochirin peptide to the subject.

20. The method of claim 19, wherein the pseudhymenochirin peptide stimulates production of pro-inflamatory cytokines.

21. The method of claim 19, wherein the pseudhymenochirin peptide supresses the production of anti-inflamatory cytokines.
